(19) United States
(12) Patent Application Publication
(10) Pub. No.: US 2019/0285459 A1
(43) Pub. Date: Sep. 19, 2019

(54) A LIQUID LEVEL MEASURING DEVICE

(71) Applicant: SMARTER TANKS PTY LTD, Bulimba, Brisbane, Queensland (AU)

(72) Inventor: Craig Anthony Aitken, Brisbane (AU)

(21) Appl. No.: 16/347,408

(22) PCT Filed: Nov. 6, 2017
(86) PCT No.: PCT/AU2017/051222

§ 371 Date: May 3, 2019

(30) Foreign Application Priority Data
Nov. 4, 2016 (AU) 2016904506

(51) Int. Cl. G01F 23/44; G01N 33/00; G01K 1/14; C12G 1/02

(52) U.S. Cl. CPC G01F 23/443; G01N 33/0044; C12G 1/02; G01K 1/14; G01N 33/004

(57) ABSTRACT

A liquid level measuring device for measuring a liquid level in a container, the device including a housing, a spool means mounted to the housing and movable during operation, a line attached to the spool means, the line windable onto and from the spool means, a float attached to the end or adjacent the end of the line, a sensor to detect a change in the tension in the line, the sensor is mounted to the housing and is operatively associated with the line, and a measuring means to measure the length of the line that is unspooled from the spool means when a predetermined change in tension in the line is detected, the predetermined change is when the float has reached the liquid level in the container, wherein in use, the line is unspooled from the spool means causing the float to move away from the housing, and wherein when a predetermined change in tension in the line is detected the measuring means measures the length of line that has unspooled from the spool means.

### FIELD OF INVENTION

The present invention relates to a liquid level measuring device. The present invention has particular but not exclusive application for measuring a liquid level in a wine tank. Reference will be made in the specification to the use of the invention with respect to wine tanks and casks. This use is by way of example only and the invention is not limited to this use.

### BACKGROUND OF THE INVENTION

In the production of wine, an accurate measurement of the amount of liquid in a tank is important. Typically a large tank is manually inspected and the level of the liquid in the tanks is determined by dipping a measuring dip stick in the tank.

Some tanks have a clear tube on the outside of the tank. The level of liquid in the clear tube shows the level of liquid in the tank.

Other tanks have laser sensors which determine the level of liquid in a tank by shining a laser onto the liquid or a float on the liquid and measuring the time it takes for the laser to return to the sensor.

A problem with using a dip stick or an outside clear tube is that the level of the liquid must be manually determined, often requiring a person to work at a height.

Another problem with a dip stick is that a person typically has to open a lid of the tank to dip the dip stick in the tank. This can introduce oxygen into a tank which can interfere with the wine fermentation process.

A problem with using clear tubes on the outside of a tank is that the tubes can become blocked with particulates in the liquid. The clear tubes can also be difficult to clean when the tanks are emptied.

The accuracy of measuring the level of liquid in a tank using laser sensors can be influenced by foam which may form on top of the liquid level.

### OBJECT OF THE INVENTION

It is an object of the present invention to overcome or at least alleviate one or more of the above problems with an alternate liquid level measuring device and/or provide the consumer with a useful or commercial choice.

### SUMMARY OF THE INVENTION

In one aspect the present invention broadly resides in a liquid level measuring device for measuring a liquid level in a container, the device including a housing;
a spool means mounted to the housing and movable during operation;

a line attached to the spool means, the line windable onto and from the spool means;

a float attached to the end or adjacent the end of the line;

a sensor to detect a change in the tension in the line, the sensor is mounted to the housing and is operatively associated with the line; and

a measuring means to measure the length of the line that is unspooled from the spool means when a predetermined change in tension in the line is detected, the predetermined change is when the float has reached the liquid level in the container, wherein in use, the line is unspooled from the spool means causing the float to move away from the housing, and wherein when a predetermined change in tension in the line is detected the measuring means measures the length of line that has unspooled from the spool means.

Preferably, the spool means includes a rotatable spool. Preferably when the spool is rotated in a first direction, the line is wound onto the spool. Preferably when the spool is rotated in a second direction, the line is unspooled from the spool.

Preferably the spool is driven by a motor. Preferably the motor is an electric motor. Preferably the electric motor is a 12 or 24 volt electric DC motor.

Preferably the line is made from non stretch nylon. More preferably the line is made from ultra high molecular weight polyethylene fibre. Preferably the line is attached at one end thereof to the spool.

Preferably the liquid level measuring device is attached to the container. Preferably the liquid level measuring device is attached to the container using one or more bolts. Preferably one of the one or more bolts has an aperture through which the line passes. Preferably the line passes into the container through the aperture of the one of the one or more bolts. Preferably the liquid level measuring device is attached to a lid or hatch of the container.

In one embodiment, the liquid level measuring device is attached near a top of the container. In the embodiment where the liquid level measuring device is attached near a top of the container, the float lowers towards the liquid level when the line is unspooled from the spool means.

In another embodiment, the liquid level measuring device is attached near a lower portion of the container. In the embodiment where the liquid level measuring device is attached near a lower portion of the container, the float floats towards the liquid level when the line is unspooled from the spool means.

In one form, the sensor includes one or more micro switches. Preferably each of the one or more micro switches is actuated when the tension in the line reaches a predetermined amount. Preferably the one or more micro switches are operatively in contact with the line. In another form, the sensor includes an accelerometer.

Preferably the accelerometer is operatively in contact with the line. Preferably a change in tension in the line causes the accelerometer to move. In another embodiment, the sensor to detect a change in the tension detects a predetermined change in the speed of movement of the spool. Typically, a change in tension in the line causes a change in the speed of movement of the spool. In one embodiment, the sensor is an encoder. In one embodiment, the measuring means utilises the encoder to measure the length of the line that is unspooled from the spool means. In another embodiment, the measuring means utilises a separate encoder to measure the length of the line that is unspooled from the spool means. Typically a sudden change in the speed of movement of the spool can indicate that the float has reached the liquid level in the container. Preferably, the length of the line that is unspooled from the spool means is recorded when the predetermined change in the speed of movement of the spool has been detected.

Preferably the measuring means is operationally associated with the spool means. Preferably the measuring means measures the amount that the spool moves during operation. Typically, the amount that the spool moves during operation correlates to the length of line that is unspooled from the spool during operation. Preferably the measuring means measures the amount that the spool rotates. Preferably the measuring means includes an encoder. Preferably the liquid level measuring device further includes a controller. Preferably the controller controls the operation of the spool means. Preferably the controller receives and processes output from the sensor. Preferably the controller receives and processes output from the measuring means. In one form, the controller processes the output from the measuring means and displays a numerical value on a display attached to the liquid level measuring device.

In another form, the controller processes the output from the measuring device and relays the output via a communication means to a remote device. Typically to determine the liquid level in the container when the liquid level measuring is located at or towards a top part of container, the remote device subtracts the length of the line that has unspooled from the spool means from a predefined distance between the bottom of the container and the liquid level measuring device.

In another form, to determine the liquid level in the container when the liquid level measuring is located at or towards a top part of container, the controller subtracts the length of the line that has unspooled from the spool means from a predefined distance between the bottom of the container and the liquid level measuring device. To determine a volume of liquid in the container, the remote device or the controller multiplies the liquid level by a predefined cross sectional area of the container. It will be appreciated that for containers that do not have a constant cross sectional area, the remote device or the controller can use appropriate formulas to determine a volume of liquid in the container based on the liquid level.

Preferably the communication means includes one or more of the following, wireless modem, wifi modem, cellular modem, network adaptor, gsm modem, 3g modem, 4g modem, and/or the like. Preferably the remote device includes one or more of the following, a server, a mobile device, a personal computer, and/or the like.

Preferably the liquid level measuring device further includes a temperature sensor to measure the temperature in the container. Preferably the temperature sensor is operatively connected to the controller. Preferably the temperature sensor is connected to the controller via a cable. Alternatively, the temperature sensor is wirelessly connected to the controller. Preferably the controller receives and processes output from the temperature sensor. In one form, the controller processes the output from the temperature sensor and displays a numerical value on a display attached to the liquid level measuring device. In another form, the controller processes the output from the temperature sensor and relays the output via a communication means to a remote device.

Preferably the liquid level measuring device further includes a humidity sensor to measure the humidity in the container. Preferably the humidity sensor is operatively connected to the controller. Preferably the humidity sensor is connected to the controller via a cable. Alternatively, the humidity sensor is wirelessly connected to the controller. Preferably the controller receives and processes output from the humidity sensor. In one form, the controller processes the output from the humidity sensor and displays a numerical value on a display attached to the liquid level measuring device. In another form, the controller processes the output from the humidity sensor and relays the output via a communication means to a remote device.

Preferably the liquid level measuring device further includes an oxygen sensor to measure a concentration of oxygen in the container. Preferably the oxygen sensor is operatively connected to the controller. Preferably the oxygen sensor is connected to the controller via a cable. Alternatively, the oxygen sensor is wirelessly connected to the controller. Preferably the controller receives and processes output from the oxygen sensor. In one form, the controller processes the output from the oxygen sensor and displays a numerical value on a display attached to the liquid level measuring device. In another form, the controller processes the output from the oxygen sensor and relays the output via a communication means to a remote device.

Preferably the liquid level measuring device further includes a CO₂ sensor to measure a concentration of CO₂ in the container. Preferably the CO₂ sensor is operatively connected to the controller. Preferably the CO₂ sensor is connected to the controller via a cable. Alternatively, the CO₂ sensor is wirelessly connected to the controller. Preferably the controller receives and processes output from the CO₂ sensor. In one form, the controller processes the output from the CO₂ sensor and displays a numerical value on a display attached to the liquid level measuring device. In another form, the controller processes the output from the CO₂ sensor and relays the output via a communication means to a remote device. In one embodiment, a tube fluidly connects the CO₂ sensor to the container. Preferably, a fan or a pump moves gas to be measured by the CO₂ sensor from the container to the CO₂ sensor.

Preferably the liquid level measuring device further includes a H₂S sensor to measure a concentration of H₂S in the container. Preferably the H₂S sensor is operatively connected to the controller. Preferably the H₂S sensor is connected to the controller via a cable. Alternatively, the H₂S sensor is wirelessly connected to the controller. Preferably the controller receives and processes output from the H₂S sensor. In one form, the controller processes the output from the H₂S sensor and displays a numerical value on a display attached to the liquid level measuring device. In another form, the controller processes the output from the H₂S sensor and relays the output via a communication means to a remote device.

Preferably the liquid level measuring device further includes an accelerometer and/or a tilt switch. When the liquid level measuring device is attached to a lid or hatch of the container, the accelerometer and/or the tilt switch can indicate whether the lid is open or closed. Preferably when the lid is open, the line is prevented from unspooling from the spool means.

In one form, the liquid level measuring device includes a mounting shim to level the liquid level measuring device on top of a lid of the container.

Preferably the liquid level measuring device further includes at least one pulley operatively associated with the line. Preferably the at least one pulley directs the line out of the liquid level measuring device. Preferably the sensor is operatively associated with the line between one of the at least one pulley and the spool means.

Preferably the liquid level measuring device further includes a cover. Preferably the cover is made of a resilient plastic. In another form, the cover is made of stainless steel. In a further form, the cover is made from a suitable metal alloy. In one embodiment, the CO₂ sensor is located within the cover.

Preferably the float is adapted to float in the liquid of which the level is being measured. Preferably the float is made of stainless steel. In another form, the float is made of a food grade plastic. In one embodiment, the float is a disc. Preferably the disc includes a plurality of apertures. The plurality of apertures are preferably located near a periphery of the disk.

Preferably the liquid level measuring device further includes a seal located between the housing and the float. Preferably the seal is located between the cover and the float. In another form, the seal is located between the float and the bolt with the aperture for the line. Preferably the seal is made of rubber. Preferably the seal is an O-ring type seal. In a further form, the seal is located between the float and a mounting nut mounted onto the bolt with the aperture for the line. Preferably the float engages with the mounting nut. Preferably a dock for the float is attached to the bolt with the aperture for the line or the mounting nut. Preferably the dock inhibits the float from excess movement when the line is wound onto the spool means.

In one embodiment, the liquid level measuring device further includes a battery to provide power to the device. In another embodiment, the liquid level measuring device is attached to an external power supply. In another form, the liquid measuring device is powered by at least one solar panel.

In a preferred embodiment, the container is a tank. Preferably the tank is a fermentation tank. More preferably the tank is a fermentation tank for wine. Preferably the liquid is wine or fermenting wine.

In one embodiment, the liquid level measuring device further includes a dissolved oxygen sensor. Preferably the dissolved oxygen sensor measures the dissolved oxygen in the liquid in the container. In one embodiment, the dissolved oxygen sensor is remote from the cover. Preferably the remote oxygen sensor extends from the cover. Preferably the dissolved oxygen sensor is operatively connected to the controller. Preferably the dissolved oxygen sensor is connected to the controller via a cable. Alternatively, the dissolved oxygen sensor is wirelessly connected to the controller. In one embodiment, the remote oxygen sensor is operatively attached to the float and moves with the float. In another embodiment, a receptacle is operatively attached to the float. Preferably the receptacle is adapted to retain a sample of the liquid when the float has reached the liquid level in the container. Preferably the dissolved oxygen sensor measures the dissolved oxygen in the sample after the line is wound onto the spool.

In one embodiment, the liquid level measuring device is at least partially located within a hatch. The hatch is preferably adapted to cover an opening of the container. In one embodiment, the hatch is substantially bowl shaped having a rim which extends downwardly in use. Preferably the hatch includes a release valve such that fluid can enter and/or exit the container. The release valve preferably opens at predetermined pressures. In one embodiment, the hatch has a release valve such that fluid can enter the container and another release valve such that fluid can exit the container.

The hatch preferably has a top surface having a solar panel or solar sheet attached thereto to provide electric power to the liquid level measuring device.

In a further aspect the present invention broadly resides in a method of measuring a liquid level in a container, including the steps of unspooling a line from a spool means, the line having a float attached to the end or adjacent the end thereof, such that the line moves towards the liquid level;

detecting with a sensor a change in the tension in the line; and

measuring with a measuring means the length of the line that is unspooled from the spool means when a predetermined change in tension is detected, the predetermined change is when the float has reached the liquid level in the container.

Preferably the method further includes the step of winding the line onto the spool means after the length of the line that is unspooled from the spool means has been measured.

Preferably the step of unspooling a line from a spool means further includes using a motor to unspool the line from the spool means. Preferably the motor is an electric motor.

Preferably the measuring means is an encoder that measures the amount that the spool means rotates.

Preferably the method further includes the step of transmitting the measured length of the line that is unspooled from the spool means to a remote device.

Preferably the method further includes the step of measuring with a temperature sensor the temperature in the container.

Preferably the method further includes the step of transmitting the measured temperature to the remote device.

Preferably the method further includes the step of measuring with a humidity sensor the humidity in the container.

Preferably the method further includes the step of transmitting the measured humidity to the remote device.

Preferably the method further includes the step of measuring with an oxygen sensor the concentration of oxygen in the container.

Preferably the method further includes the step of transmitting the measured oxygen concentration to the remote device.

Preferably the method further includes the step of measuring with a dissolved oxygen sensor the concentration of dissolved oxygen in the liquid.

Preferably the method further includes the step of transmitting the measured dissolved oxygen concentration to the remote device.

Preferably the method further includes the step of measuring with a CO₂ sensor the concentration of CO₂ in the container.

Preferably the method further includes the step of transmitting the measured CO₂ concentration to the remote device.

Preferably the method further includes the step of measuring with a H₂S sensor the concentration of H₂S in the container.

Preferably the method further includes the step of transmitting the measured H₂S concentration to the remote device.

In another aspect, the invention broadly resides in a tank for storing a liquid, the tank including:

a liquid level measuring device for measuring a liquid level in the tank, the device including

a housing;

a spool means mounted to the housing and movable during operation;

a line attached to the spool means, the line windable onto and from the spool means;

a float attached to the end or adjacent the end of the line;

a sensor to detect a change in the tension in the line, the sensor is mounted to the housing and is operatively associated with the line; and

a measuring means to measure the length of the line that is unspooled from the spool means when a predetermined change in tension in the line is detected, the predetermined change is when the float has reached the liquid level in the tank.

Preferably the tank is a stainless steel tank. Preferably the tank is a fermentation tank. More preferably the tank is a fermentation tank for wine. Alternatively, the tank can be a storage tank for wine.

Preferably the liquid level measuring device is attached to a lid or hatch of the tank.

The features described with respect to one aspect also apply where applicable to all other aspects of the invention. Furthermore, different combinations of described features are herein described and claimed even when not expressly stated.

### BRIEF DESCRIPTION OF THE DRAWINGS

In order that the present invention can be more readily understood reference will now be made to the accompanying drawings which illustrate a preferred embodiment of the invention and wherein:

FIG. 1A is a rear perspective view of a liquid measuring device according to an embodiment of the present invention;

FIG. 1B is a plan view of the liquid measuring device of FIG. 1A;

FIG. 10 is a left side view of the liquid measuring device of FIG. 1A;

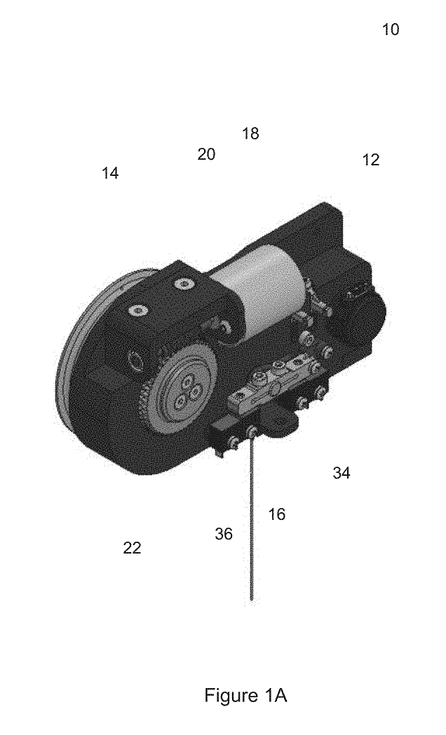

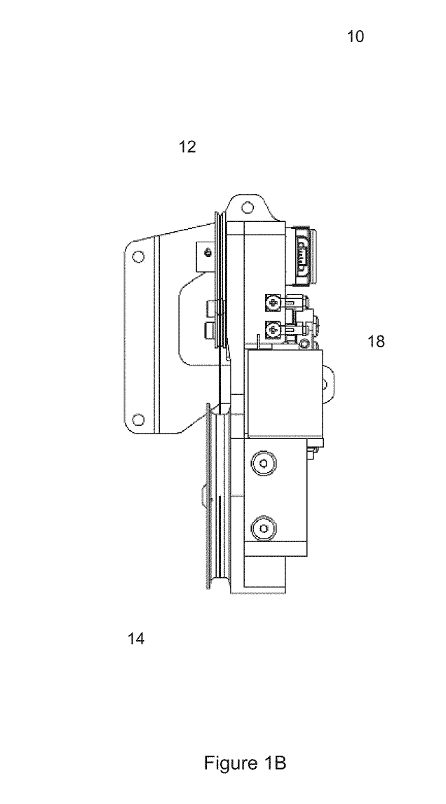

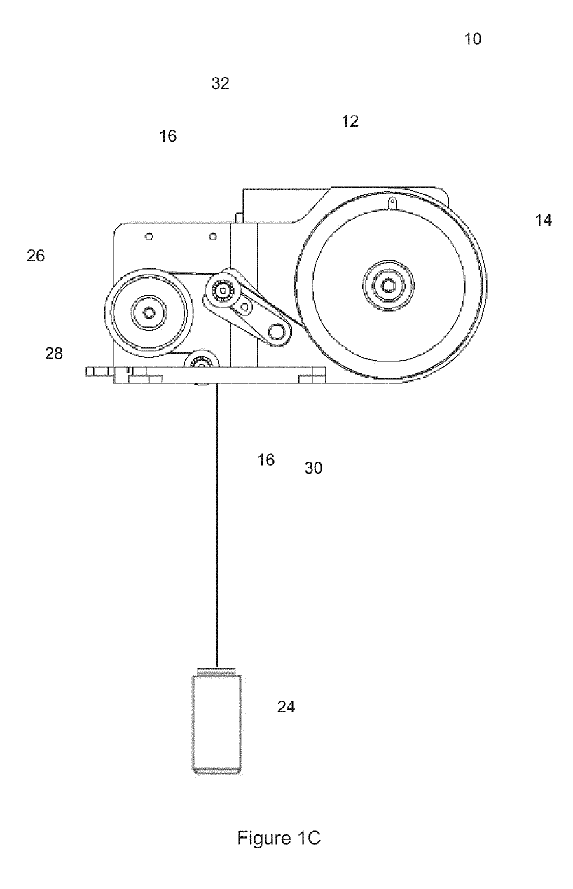

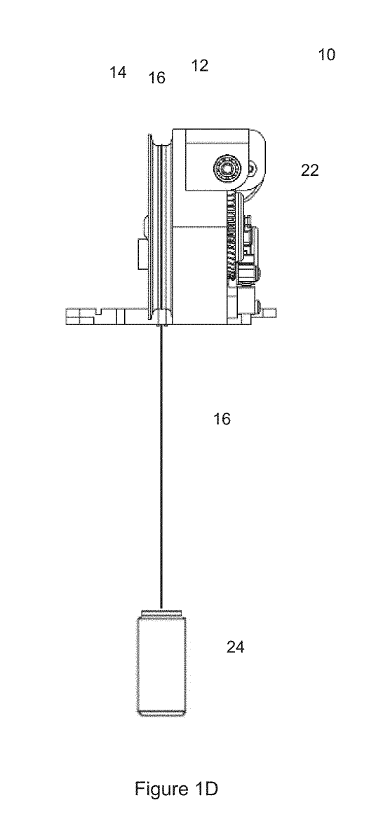

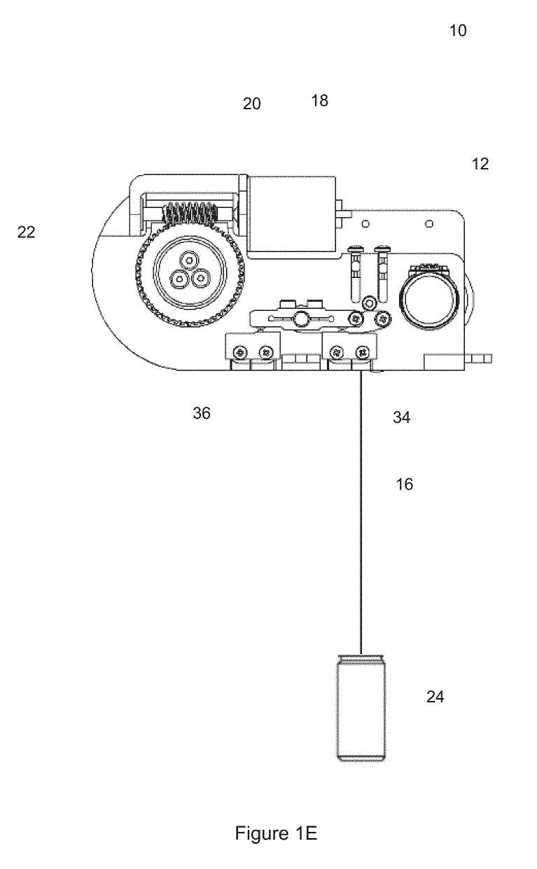

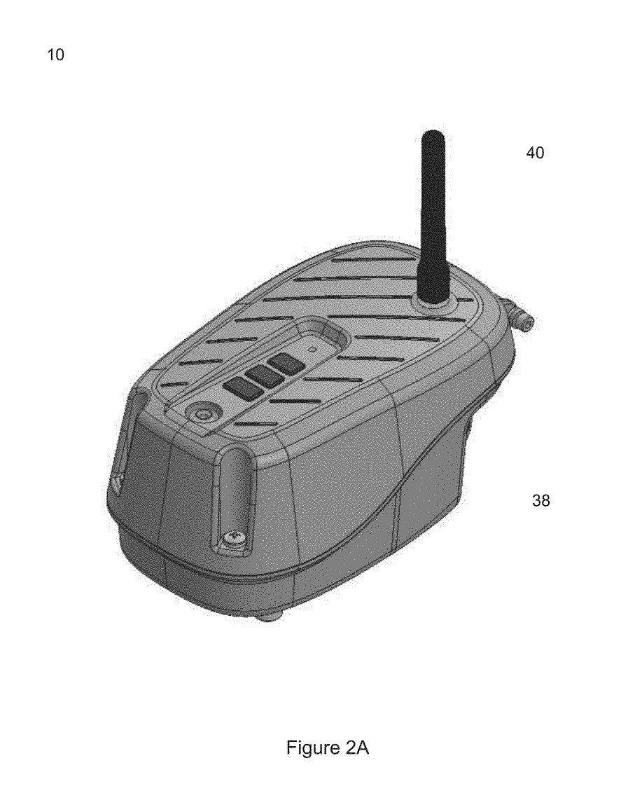

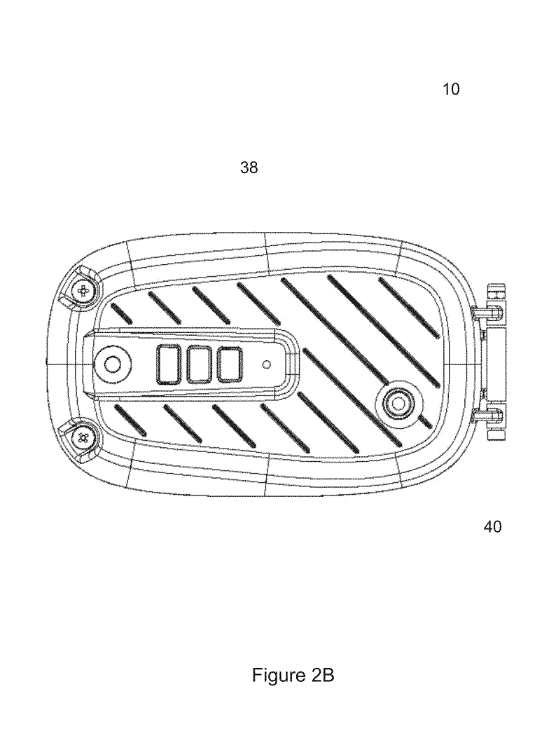

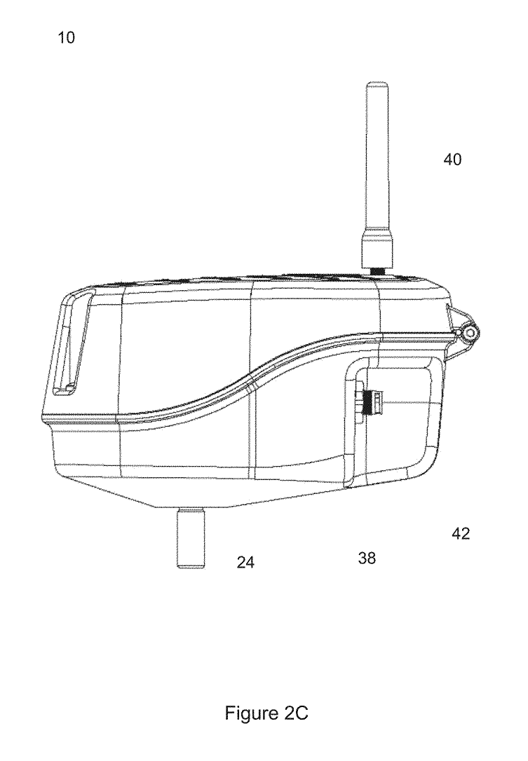

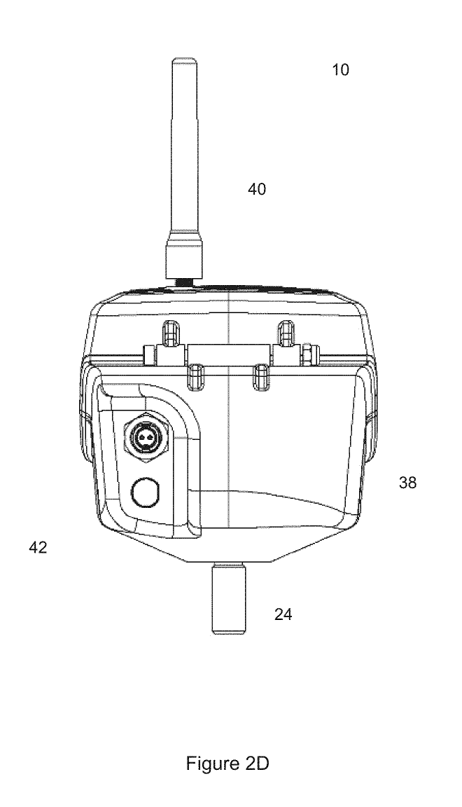

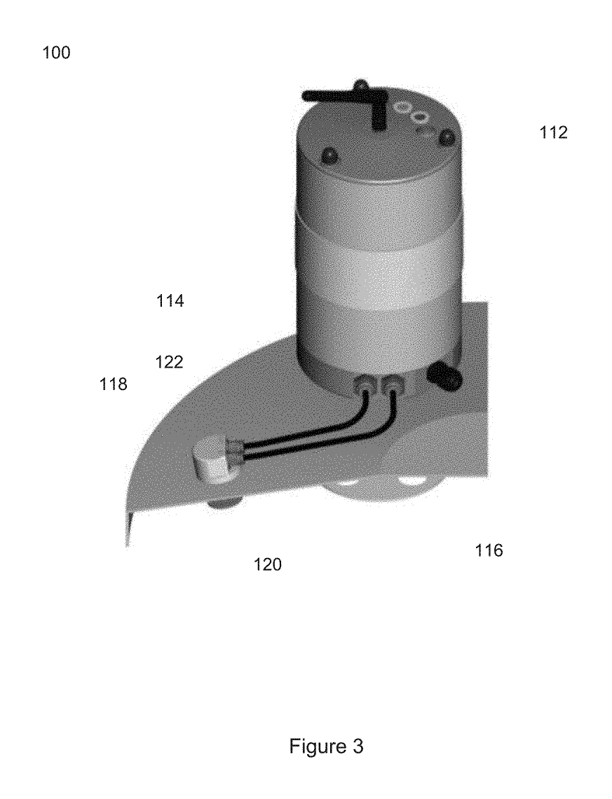

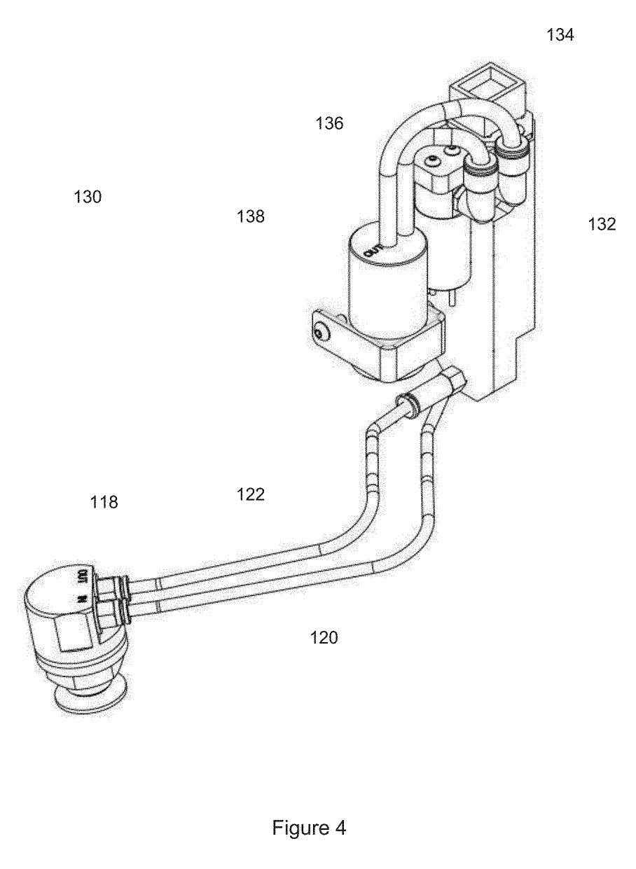

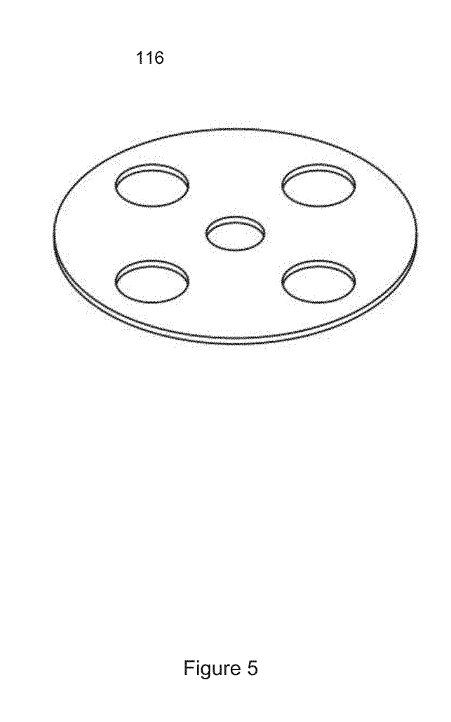

FIG. 1D is a rear view of the liquid measuring device of FIG. 1A;

FIG. 1E is a right side view of the liquid measuring device of FIG. 1A;

FIG. 2A is a perspective view of a liquid measuring device having a cover;

FIG. 2B is a plan view of the liquid measuring device of FIG. 2A;

FIG. 2C is a left side view of the liquid measuring device of FIG. 2A;

FIG. 2D is a rear view of the liquid measuring device of FIG. 2A;

FIG. 3 is a perspective partially sectioned view of a liquid measuring device, according to another embodiment of the present invention, attached to a hatch of a container;

FIG. 4 is perspective view of a CO₂ sensor assembly of the liquid measuring device of FIG. 3; and

FIG. 5 is a perspective view of the disc float of the liquid measuring device of FIG. 3.

### DETAILED DESCRIPTION OF THE PREFERRED EMBODIMENT

With reference to FIGS. 1A to 1E there is shown a liquid measuring device 10 according to an embodiment of the present invention. The liquid measuring device 10 has a housing 12. A spool means in the form of a spool 14 is mounted to the housing 12. A line 16 is attached to the spool 14. The line 16 is windable onto and from the spool 14. The spool 14 is driven by an electric motor 18 via a worm gear 20 and a gear 22 which is attached to the spool 14.

A float 24 is attached to the end of the line 16. The float 24 is made from stainless steel and is hollow so that it can float in liquid.

The liquid measuring device 10 has pulleys 26, 28 which help guide the line 16.

The liquid measuring device 10 has a sensor 30 to detect a change in the tension in the line 16. The sensor 30 has a pulley 32 which engages with the line 16. The sensor 30 is pivotably mounted to the housing 12 and biased so that the pulley 32 engages with the line 16. When the tension in the line 16 changes, the sensor 30 pivots. A micro switch 34 associated with the sensor 30 engages when the tension in the line 16 increases by a predetermined amount and another micro switch 36 associated with the sensor 30 engages when the tension in the line 16 decreases by a predetermined amount.

The liquid measuring device 10 has a measuring means in the form of an encoder (not shown) associated with the spool 14. The encoder (not shown) measures the number of times that the spool 14 rotates, this equates to the length of the line 16 that is unspooled from the spool 14.

The liquid measuring device 10 also includes a controller (not shown) which receives output signals from the micro switches 34,36 and the encoder (not shown). The controller (not shown) controls the electric motor 18 to control the rotation of the spool 14.

With reference to FIGS. 2A to 2D there is shown a liquid measuring device 10 having a cover 38. The cover 38 is made from stainless steel and protects the internal parts of the liquid measuring device 10.

The cover 38 also enables the liquid measuring device 10 to be attached to a lid of a container (not shown).

The liquid measuring device 10 has an antenna 40 which enables the controller (not shown) to communicate with a remote server (not shown).

The liquid measuring device 10 has a connector 42. The connector 42 enables the liquid measuring device 10 to be connected to an external power supply (not shown). The connector 42 also enables the liquid measuring device 10 to be connected to sensors (not shown) such as a temperature sensor, humidity sensor, oxygen sensor, and/or a CO₂ sensor. The liquid measuring device 10 can relay the readings from the sensors (not shown) to the remote server (not shown)

In use, with reference to FIGS. 1A to 2D, initially the float 24 is located adjacent the cover 38 (as seen in FIGS. 2A to 2D). When the controller (not shown) is sent a signal from a remote server (not shown) to measure the liquid level of a tank (not shown) to which the liquid measuring device 10 is attached, the controller engages the electric motor 18 to rotate the spool 14 so that the line 16 is unspooled from the spool 14.

As the line 16 is being unspooled from the spool 14 and the float 24 is moving away from the housing 12 of the liquid measuring device 10, the sensor 30 which is biased towards and engages with the line 16 detects the tension in the line 16. As the line 16 is being unspooled from the spool 14, the encoder (not shown) measures the number of times that the spool 14 rotates. When the float 24 reaches the liquid level of the tank (not shown), the tension in the line 16 is reduced. The reduction in the tension of the line causes the sensor 30 to pivot, which causes micro switch 36 to activate. On receiving the activation signal (output signal) from the micro switch 36, the controller (not shown) stops the electric motor 18. The controller (not shown) relays the output from the encoder (not shown) to the remote server (not shown). The remote server (not shown) converts the output from the encoder to a length of the line 16 that has unspooled from the spool means 14. To determine the level of the liquid in the tank (not shown), the remote server (not shown) subtracts the length of the line 16 that has unspooled from the spool means 14 from a predefined distance between the bottom of the tank and the liquid level measuring device 10.

After receiving the activation signal (output signal) from the micro switch 36, the controller (not shown) activates the electric motor 18 to rotate the spool 14 so that the line 16 is wound onto the spool 14. As the line 16 is being wound onto the spool 14 and the float 24 is moving towards the housing 12 of the liquid measuring device 10, the sensor 30 which is biased towards and engages with the line 16 detects the tension in the line 16. When the float 24 abuts the cover 38, the tension in the line 16 increases. The increase in the tension of the line causes the sensor 30 to pivot, which causes the micro switch 34 to activate. On receiving the activation signal (output signal) from the micro switch 34, the controller (not shown) stops the electric motor 18.

With reference to FIG. 3 there is shown a liquid measuring device 100 according to another embodiment of the present invention. The liquid measuring device 100 has a housing 112. The housing 112 is attached to a hatch 114 of a container (not shown). The hatch 114 is shown as partially sectioned. A line (not shown) extends from the housing 112 through the surface of the hatch 114. A float in the form of a flat disc 116 is attached to the line (not shown). An inlet/outlet portion 118 of a CO₂ sensor assembly (best seen in FIG. 4) is attached to the hatch 114 and remote from the housing 112. Part of the inlet/outlet portion 118 extends through the hatch 114 so that air to be sampled is drawn from and returned to below the hatch 114. Inlet tube 120 and outlet tube 122 extend from the inlet/outlet portion 118 into the housing 112.

With reference to FIG. 4, there is shown a CO₂ sensor assembly 130. The inlet/outlet portion 118 is connected to a manifold 132 by inlet tube 120. A CO₂ sensor 134 is in fluid communication with the manifold 132 and measures the CO₂ concentration of the gas that passes through the manifold 132. The CO₂ sensor assembly 130 further includes a solenoid valve 136 and a pump 138 to move the gas from the inlet outlet portion 118 through the inlet tube 120, through the manifold 132 so that the gas can be measured by the CO₂ sensor 134, and back towards the inlet outlet portion 118 through the outlet tube 122.

With reference to FIG. 5, there is shown a detailed view of the disc 116. The disc 116 is not designed to float in a liquid in a container (not shown), instead, when the disc 116 reaches the liquid level of the container (not shown), the tension in the line (not shown) is reduced, which can be detected as a reduction in the tension of the line and/or a reduction in the speed of the line.

### Advantages

An advantage of the preferred embodiment of the liquid level measuring device includes that the level of liquid in a container can be established without manual intervention. Another advantage of the preferred embodiment of the liquid level measuring device includes that level of liquid in a container can be established for liquids that are covered by a layer of foam. An advantage of including CO₂ and H₂S sensors is that measuring CO₂ and H₂S levels can provide an indication of whether a wine tank requires dry icing to reduce oxygen levels with an intent to minimise the risk of harmful bacteria growing in the tank. An advantage of including an accelerometer and/or a tilt switch is that the accelerometer and/or the tilt switch can assist in determining if the lid of the container has inadvertently been left open. An advantage of detecting a predetermined change in the speed of movement of the spool is that the float does not need to be buoyant.

### Variations

While the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1. A liquid level measuring device for measuring a liquid level in a container, the device including a housing;
a spool means mounted to the housing and movable during operation;
a line attached to the spool means, the line windable onto and from the spool means;
a float attached to the end or adjacent the end of the line;
a sensor to detect a change in the tension in the line, the sensor is mounted to the housing and is operatively associated with the line; and
a measuring means to measure the length of the line that is unspooled from the spool means when a predetermined change in tension in the line is detected, the predetermined change is when the float has reached the liquid level in the container,
wherein in use, the line is unspooled from the spool means causing the float to move away from the housing, and wherein when a predetermined change in tension in the line is detected the measuring means measures the length of line that has unspooled from the spool means.

2. A liquid level measuring device as claimed in claim 1, wherein the liquid level measuring device is attached near a top of the container and the float lowers towards the liquid level when the line is unspooled from the spool means.

3. A liquid level measuring device as claimed in claim 1, wherein the liquid level measuring device is attached near a lower portion of the container and the float floats towards the liquid level when the line is unspooled from the spool means.

4. A liquid level measuring device as claimed in claim 1, wherein the sensor includes one or more micro switches actuated when the tension in the line reaches a predetermined amount, or an accelerometer operatively in contact with the line, to detect a change in the tension in the line.

5. A liquid level measuring device as claimed in claim 1, wherein the sensor detects a predetermined change in the speed of movement of the spool to detect a change in the tension in the line.

6. A liquid level measuring device as claimed in claim 1, wherein the liquid level measuring device is attached to a lid or hatch of the container.

7. A liquid level measuring device as claimed in claim 6, wherein the liquid level measuring device further includes an accelerometer and/or a tilt switch to indicate whether the lid or hatch is open or closed, and prevent the line from unspooling from the spool means when the lid or hatch is open.

8. A liquid level measuring device as claimed in claim 1, further including a temperature sensor to measure the temperature in the container, and a humidity sensor to measure the humidity in the container.

9. A liquid level measuring device as claimed in claim 1, further including an oxygen sensor to measure a concentration of oxygen in the container, and a dissolved oxygen sensor to measure the dissolved oxygen in the liquid in the container.

10. A liquid level measuring device as claimed in claim 1, further including a CO₂ sensor to measure a concentration of CO₂ in the container, and a H₂S sensor to measure a concentration of H₂S in the container.

11. A liquid level measuring device as claimed in claim 1, wherein the liquid level measuring device is at least partially located within a hatch adapted to cover an opening of the container

12. A method of measuring a liquid level in a container, including the steps of
unspooling a line from a spool means, the line having a float attached to the end or adjacent the end thereof, such that the line moves towards the liquid level;
detecting with a sensor a change in the tension in the line; and
measuring with a measuring means the length of the line that is unspooled from the spool means when a predetermined change in tension is detected, the predetermined change is when the float has reached the liquid level in the container.

13. A method as claimed in claim 12, further including the step of winding the line onto the spool means after the length of the line that is unspooled from the spool means has been measured.

14. A method as claimed in claim 12, further including the step of measuring with a temperature sensor the temperature in the container, and measuring with a humidity sensor the humidity in the container.

15. A method as claimed in claim 12, further including the step of measuring with an oxygen sensor the concentration of oxygen in the container, and measuring with a dissolved oxygen sensor the concentration of dissolved oxygen in the liquid.

16. A method as claimed in claim 12, further including the step of measuring with a CO₂ sensor the concentration of CO₂ in the container, and measuring with a H₂S sensor the concentration of H₂S in the container.

17. A method as claimed in claim 12, further including the step of transmitting the measurement(s) to a remote device.

18. A tank for storing a liquid, the tank including
a liquid level measuring device for measuring a liquid level in the tank, the device including
a housing;
a spool means mounted to the housing and movable during operation;
a line attached to the spool means, the line windable onto and from the spool means;
a float attached to the end or adjacent the end of the line;
a sensor to detect a change in the tension in the line, the sensor is mounted to the housing and is operatively associated with the line; and
a measuring means to measure the length of the line that is unspooled from the spool means when a predetermined change in tension in the line is detected, the predetermined change is when the float has reached the liquid level in the tank.

19. A tank as claimed in claim 18, wherein the tank is a stainless steel tank.

20. A tank as claimed in claim 18, wherein the tank is a fermentation tank for wine or a storage tank for wine.
